(19) United States
(12) Patent Application Publication
(10) Pub. No.: US 2007/0106166 A1
(43) Pub. Date: May 10, 2007

(54) Apparatuses for ensuring positioning of a blood pressure cuff and methods of using the same

(76) Inventor: Benjamin L. Somberg, Shoreview, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/248,394

(22) Filed: Oct. 12, 2005

(51) Int. Cl. A61B 5/02

(52) U.S. Cl. 600/499; 600/490

(57) ABSTRACT

Apparatuses and methods ensure positioning of a blood pressure cuff on an arm to affect accurate measurements of blood pressure. An apparatus includes an elongated air bag of predetermined length disposed in the blood pressure cuff and adapted for inflation. The length has opposite ends to be placed around the arm. The apparatus also includes an appendage affixed to the cuff and extended from a bottom edge of the cuff. The appendage is affixed in a position relative to the cuff and formed in shape to fit an elbow in a manner that ensures the cuff is positioned to affect an accurate measurement of the blood pressure. Additionally the apparatus includes a tube attached to the cuff. When the cuff is positioned around the arm and the appendage is positioned on the elbow, a measuring end of the tube is forced into position near a brachial artery of the patient.

### TECHNICAL FIELD

The present invention relates to blood pressure measurements. More particularly, but not exclusively, the present invention relates to apparatuses and methods for ensuring proper placement of blood pressure cuffs during measurement.

### BACKGROUND

In order for home health monitoring systems to consistently diagnose health issues with accuracy, patient self-measurements of vitals, for instance blood pressure measurements, must also be accurate. Usability testing in which participants had to use home blood pressure monitors revealed that participants who were given instructions on where to place the blood pressure cuff in order to obtain an accurate reading, often failed to position the cuff correctly. The bottom of the cuff should be placed about one inch above the elbow and a measuring end of the tube attached to the cuff should rest across the inside of the elbow. Because of the errors in positioning the blood pressure cuff, there was a substantial variability in the blood pressure readings obtained via the participants' self measurements.

Despite extensive user training for use of blood pressure measurement devices, self-measurements are still prone to inaccuracy and variability due to flawed measurement practices by users. Users also become frustrated with the training and measurement process. Some physicians have reported skepticism about patient self-monitoring systems unless they can be assured of the reliability and validity of measurements.

Accordingly there is an unaddressed need in the industry to address the aforementioned and other deficiencies and inadequacies.

### SUMMARY

This Summary is provided to introduce a selection of concepts in simplified form that are further described below in the Detailed Description. This Summary is not intended to identify key features or essential features of the claimed subject matter, nor is the Summary intended for use as an aid in determining the scope of the claimed subject matter.

In accordance with the present invention, the above and other problems are solved by embodiments of the present invention. Embodiments of the present invention are directed to apparatuses and methods for ensuring proper positioning of a blood pressure cuff on an arm of a patient to affect an accurate measurement of blood pressure. Embodiments of the present invention help to ensure accurate and reliable readings by placing the blood pressure cuff in a proper position for accurate blood pressure measurements. Additionally, embodiments of the present invention reduce the need for user training and will likely help avoid user frustration with the measurement process.

One embodiment is a blood pressure cuff apparatus for ensuring proper positioning of a blood pressure cuff on an arm of a patient to affect an accurate measurement of blood pressure. The blood pressure cuff apparatus includes an elongated air bag of predetermined length disposed in the blood pressure cuff and adapted for inflation. The predetermined length has opposite ends to be placed around the arm of a patient. The apparatus also includes an appendage affixed to the cuff and extended from a bottom edge of the cuff between the opposite ends. The appendage is affixed in a position relative to the cuff and formed in shape to fit upon an elbow of a patient. The appendage fits the elbow in a manner that ensures that the cuff is positioned on the arm of the patient to affect an accurate measurement of the blood pressure. The apparatus still further includes a tube having a measurement end attached to the cuff. Thus, when the cuff is positioned around the arm and the appendage is positioned on the elbow of the patient, the measurement end of the tube is forced into a position relative to a brachial artery of the patient to influence an accurate measurement of blood pressure.

Another embodiment is an apparatus for ensuring a positioning of a blood pressure cuff on an arm of a patient to affect an accurate measurement of blood pressure by a blood pressure monitor used in conjunction with the blood pressure cuff. The apparatus is an appendage affixed to the cuff and extended from a bottom edge of the cuff between opposite ends of the cuff. The appendage is affixed in a position relative to the cuff and formed in a shape to complementarily receive an elbow of the patient in a manner that ensures that one or more elements of the cuff are forced in a position that enables accurate measurement of blood pressure. The elements of the cuff include a tube having a measuring end to be placed near a brachial artery of the patient in order for the blood pressure monitor to accurately measure the blood pressure of the patient.

Still another embodiment is a method for ensuring a position of a blood pressure cuff on an arm of a patient to affect an accurate measurement of blood pressure. The method involves providing a blood pressure cuff apparatus. The apparatus includes an elongated air bag adapted to be inflated and having opposite ends disposed in the blood pressure cuff to be placed around the arm of the patient. The apparatus also includes an appendage affixed to the cuff and extended from a bottom edge of the cuff between the opposite ends. The appendage is affixed in a position relative to the cuff and formed in a shape to fit upon an elbow of the patient in a manner that ensures that the cuff is positioned on the arm of the patient to affect an accurate measurement of the blood pressure. Additionally, the apparatus includes a tube having a measuring end attached to the cuff. When the cuff is positioned around the arm and the appendage is positioned on the elbow of the patient, the measuring end of the tube is forced into a position near a brachial artery of the patient.

The method also involves placing the appendage over the elbow of the patient where the appendage complementarily receives the elbow and placing the cuff around the arm of the patient. Thus, proper placement of the cuff to affect an accurate measurement of blood pressure is ensured.

These and other features and advantages, which characterize the present invention, will be apparent from a reading of the following detailed description and a review of the associated drawings. It is to be understood that both the foregoing general description and the following detailed description are exemplary and explanatory only and are not restrictive of the invention as claimed.

### BRIEF DESCRIPTION OF THE DRAWINGS

FIG. 1 is a frontal isometric view of a blood pressure cuff apparatus shown in the open position according to an illustrative embodiment of the present invention;

FIG. 2 is a rear isometric view of the blood pressure cuff apparatus illustrated in FIG. 1 in the open position according to an illustrative embodiment of the present invention;

FIG. 3 is an isometric view of a blood pressure cuff apparatus illustrated receiving an upper arm and elbow of a patient for ensuring positioning to affect an accurate blood pressure determination according to an illustrative embodiment of the present invention;

FIG. 4 is a frontal isometric view of an alternative illustrative embodiment of the blood pressure cuff apparatus; and

FIG. 5 is a rear isometric view of the alternative illustrative embodiment of the blood pressure cuff apparatus illustrated in FIG. 4.

### DETAILED DESCRIPTION

As described briefly above, embodiments of the present invention are directed to apparatuses and methods for ensuring proper positioning of a blood pressure cuff on an arm of a patient to affect an accurate measurement of blood pressure. In the following detailed description, references are made to the accompanying drawings that form a part hereof, and in which are shown by way of illustrations, specific embodiments or examples. These embodiments may be combined, other embodiments may be utilized, and structural changes may be made without departing from the spirit or scope of the present invention. The following detailed description is therefore not to be taken in a limiting sense and the scope of the present invention is defined by the appended claims and their equivalents.

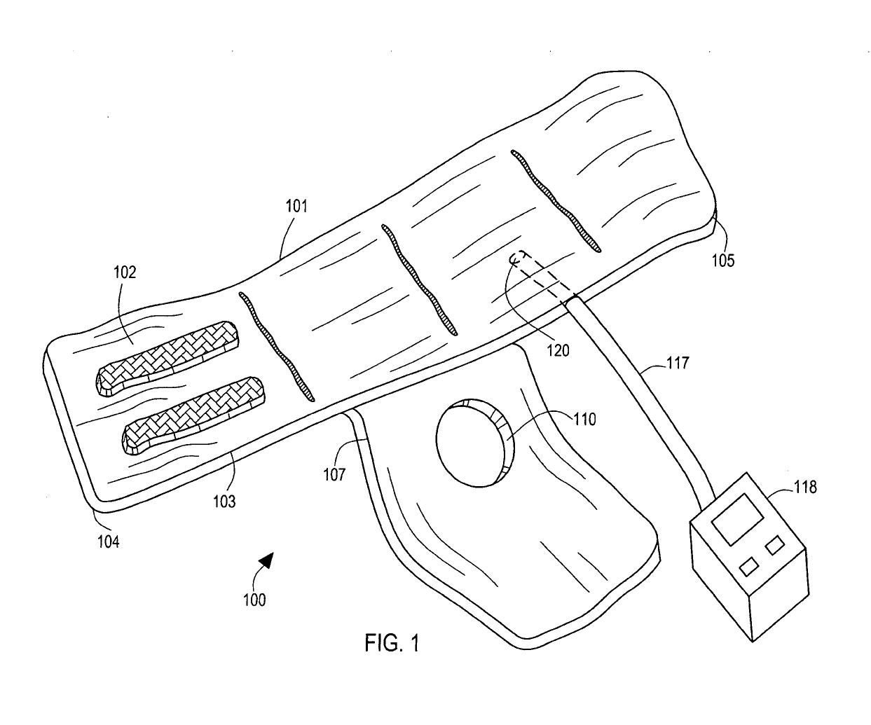

For purposes of the following description, the terms “top”, “bottom”, “front” and “rear” and relative terms of similar reference shall refer to the orientation of embodiments of the invention as shown in FIGS. 1 through 5, except where expressly specified to the contrary. Specific dimensions and other physical characteristics related to different embodiments are not to be considered as limiting unless the claims expressly state otherwise.

FIG. 1 is a frontal isometric view of a blood pressure cuff apparatus (BPCA) 100 shown in the open position according to an illustrative embodiment of the present invention. The BPCA 100 includes an elongated flexible air bag 102 of predetermined length disposed in a blood pressure cuff 101 with inner and outer surface layers and adapted for inflation. The predetermined length has opposite ends 104 and 105 to be placed around the arm of a patient. The air bag 102 has a plurality of air-impermeable compartments (not shown) between the inner and outer surface layers that can be connected to a pressurization source to allow the air bag 102 to be inflated prior to a blood pressure measurement. The blood pressure cuff 101 may be formed of layers of a durable woven fabric such as nylon, and the air impermeable compartments located between the inner and outer layers may be retained in position by stitching.

Alternatively, other structures could be used to form the blood pressure cuff 101, such as fabricating from rubber-impregnated fabrics, or from entirely non-woven resilient materials. The blood pressure cuff 101 may also be fabricated as a single structure, with the air impermeable compartments formed within the band.

The BPCA 100 also includes an appendage 107 affixed to the cuff 101 and extended from a bottom edge 103 of the cuff 101 between the opposite ends 104 and 105. The appendage 107 is affixed in a position relative to the cuff 101 and formed in a shape to fit upon an elbow of a patient in a manner that ensures that the cuff 101 is positioned on the arm of the patient to affect or influence an accurate measurement of blood pressure.

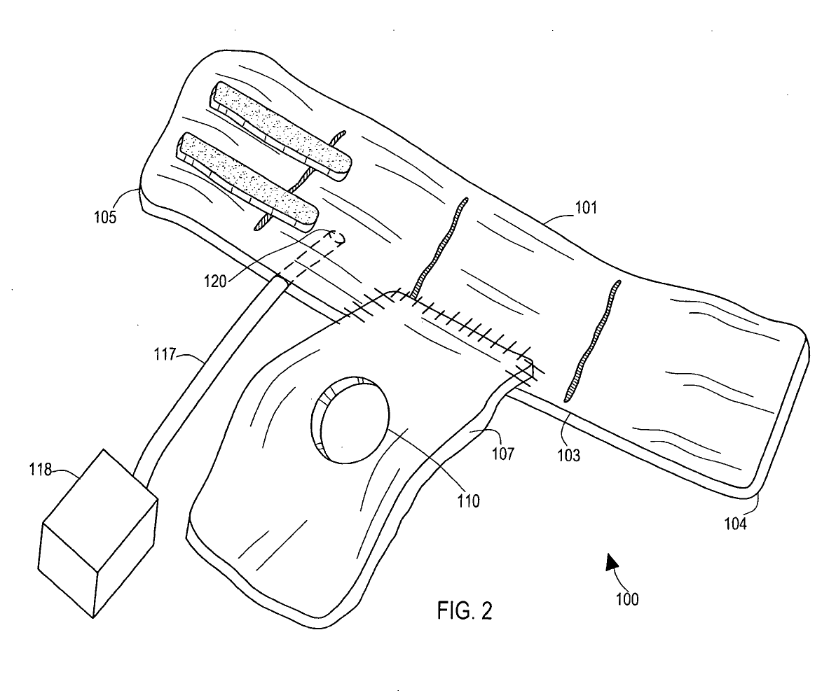

The appendage 107 includes an indented and/or cut out region 110 formed to complementarily receive the elbow of a patient. The appendage 107 may be affixed in the position relative to the cuff 101 to fit upon the elbow of the patient based upon anthropometric data that enables accurate placement of the blood pressure cuff for a significant number of patients. Affixing of the appendage 107 may alternatively be based upon a one-time placement by a health professional to match or accommodate an arm size of the patient. Still further, affixing the appendage 107 may be based on predetermined sizes for the BPCA 100. For instance, a manufacturer may produce a number of sizes based on variety of circumferences, diameters and/or lengths of arms for a potential user population. The appendage can be made of semi-flexible material such as plastic or foam rubber and covered in nylon for comfort. Additional details regarding affixing the appendage to the cuff 101 will be described below with respect to FIG. 2.

Still further, the BPCA 100 includes a tube 117 having a measurement end 120 attached to the cuff 101. The tube is also attached to a blood pressure monitoring device 118. When the cuff 101 is positioned around the arm and the appendage 107 is positioned on the elbow of the patient, the measurement end 120 of the tube 117 is forced into a position relative to a brachial artery of the patient that enables a most accurate measurement of blood pressure.

FIG. 2 is a rear isometric view of the BPCA 100 illustrated in FIG. 1 in the open position according to an illustrative embodiment of the present invention. The appendage 107 may be permanently affixed to the cuff 101 by permanent stitching. In the alternative, the appendage 107 may be detachably affixed to the cuff 101 with Velcro, metal clamps, one or more straps, or hooks. As can be seen by the rear view, the cutout region 110 formed to receive the elbow of a patient goes all the way through the appendage 107. Typically when the appendage 107 is placed over the elbow, positioning of the bottom edge 103 of the cuff 101 around the arm is a distance above the elbow of the patient, for example approximately one inch, for the most accurate blood pressure readings. Thus, as described above, the BPCA 100 ensures positioning of the blood pressure cuff 101 on the arm of a patient to affect an accurate measurement of blood pressure. For example, the blood pressure monitoring device 118 used in conjunction with the blood pressure cuff 101 may measure the blood pressure.

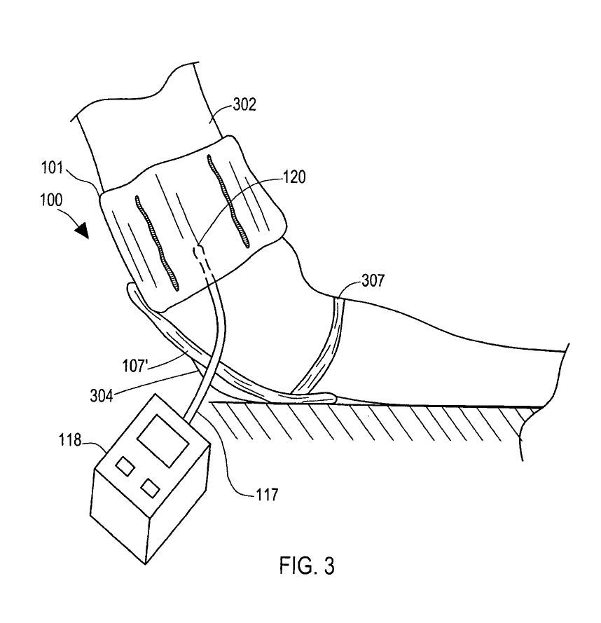

FIG. 3 is an isometric view of a BPCA 100′ receiving an upper arm 302 and an elbow 304 of a patient for ensuring positioning to affect an accurate blood pressure determination according to an illustrative embodiment of the present invention. As shown in FIG. 3, the cuff 101 surrounds the upper arm 302 while the elbow 304 naturally fits on an appendage 107′ to ensure proper cuff 101 and tube 117 placements for the most accurate blood pressure readings. The appendage 107′ also includes a strap 307 attached to the appendage to assist in holding the appendage 107′ in place. When the elbow 304 is placed in the appendage 107′, the measuring end 120 of the tube 117 is positioned near the brachial artery of the arm 302. The tube 117 rests across the inside of the elbow 304 according to standard instructions given to test subjects or patients.

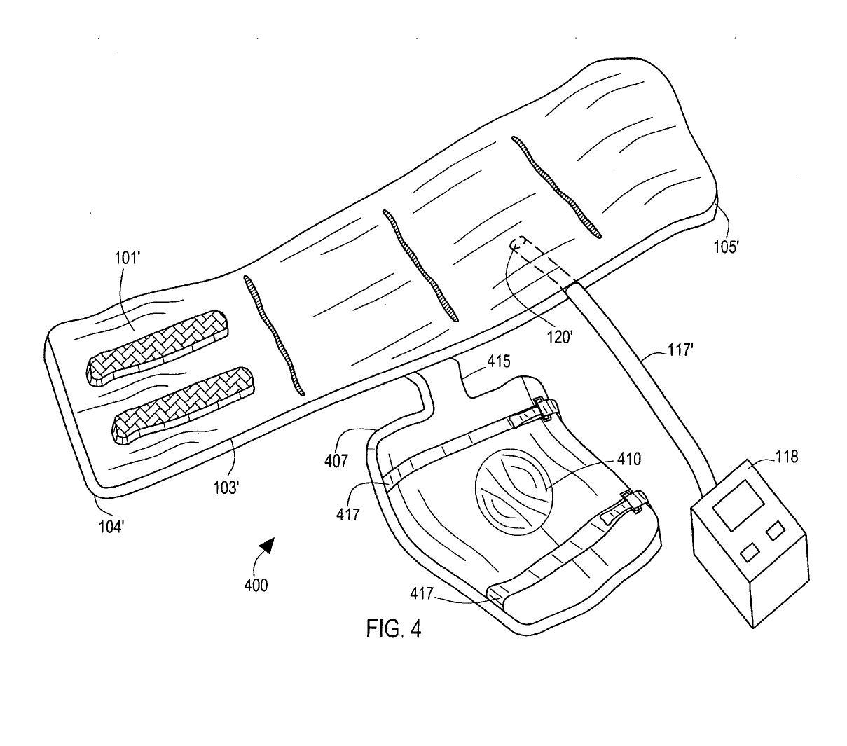

Referring now to FIG. 4, a frontal isometric view of an alternative illustrative embodiment of a BPCA 400 will be described. In this embodiment, the BPCA 400 includes an appendage 407 with an indented region 410. The indented region 410 allows the appendage to complementarily receive an elbow when placing a cuff 101′ around an arm of a test subject. The indented region 410 affords the test subject additional comfort by cushioning the elbow versus the cutout embodiment. The appendage 407 also includes straps 417 attached to the appendage 407 to assist in holding the appendage 407 in place with respect to an arm of the patient. The appendage 407 also includes a narrower extended region 415 for use in affixing the appendage 407 to the cuff 101′. As briefly described above the appendage 407 with the extended region 415 is made of a rigid or semi-rigid material, for example hard plastic or foam. As in the previous embodiment, placement of an elbow in the appendage 407 will ensure proper placement of the cuff 101′ around the arm for the most accurate blood pressure readings.

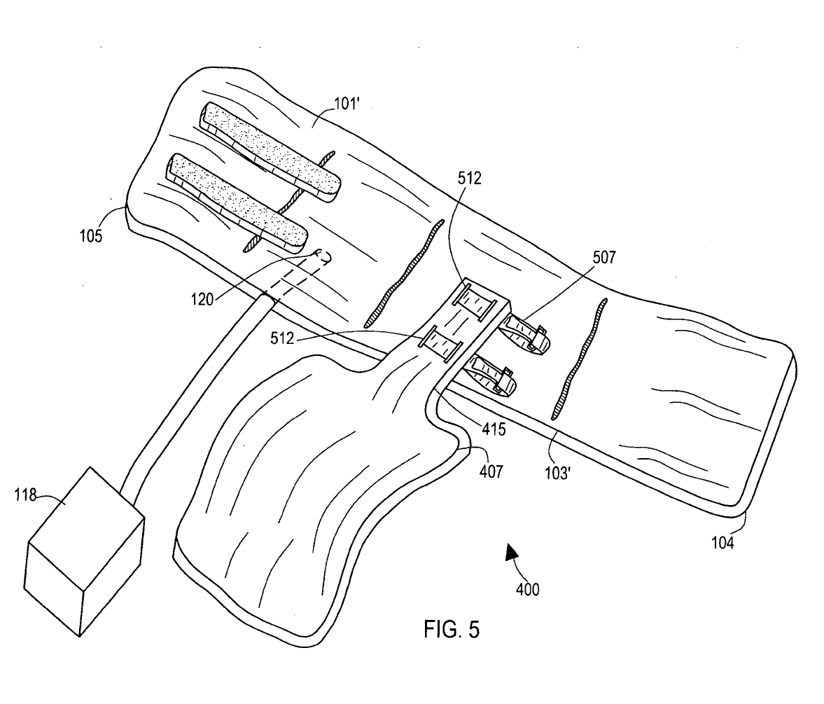

FIG. 5 is a rear isometric view of the alternative illustrative embodiment of the BPCA 400 of FIG. 4. The appendage 407 may be permanently affixed to the cuff 101′ by permanent stitching. In the alternative, the appendage 407 can be detachably affixed to the cuff 101′ with straps, Velcro, clamps, or hooks. As illustrated in FIG. 5, straps 507, permanently affixed to the cuff 101′, detachably affix the appendage 407 to the cuff 101′. The straps 507 are threaded through open slots 512 in the appendage 407. The straps 507 are made of a flexible material, for example nylon.

Also, as can be seen by the rear view, the indented region 410 (FIG. 4) formed to receive the elbow of a test subject does not show through the bottom of the appendage 407 thus, providing added cushioning for the elbow. Typically when the appendage 407 is placed over the elbow, positioning of a bottom edge 103′ of the cuff 101′ around the arm is a distance, for instance approximately one inch, above the elbow of the patient or test subject for the most accurate blood pressure readings.

Thus, the present invention is presently embodied as apparatuses and methods for ensuring proper positioning of a blood pressure cuff on an arm of a patient to affect an accurate measurement of blood pressure. The above specification, examples and data provide a complete description of the manufacture and use of the composition of the invention. Since many embodiments of the invention can be made without departing from the spirit and scope of the invention, the invention resides in the claims hereinafter appended.

We claim:

1. A blood pressure cuff apparatus for ensuring proper positioning of a blood pressure cuff on an arm of a patient to affect an accurate measurement of blood pressure, the blood pressure cuff apparatus comprising:
an elongated air bag of predetermined length disposed in the blood pressure cuff and adapted to be inflated, the length having opposite ends to be placed around the arm of a patient;
an appendage affixed to the cuff and extended from an edge of the cuff between the opposite ends, the appendage affixed in a position relative to the cuff and formed in shape to fit upon an elbow of a patient in a manner that ensures that the cuff is positioned on the arm of the patient to affect an accurate measurement of the blood pressure; and
a tube having a measurement end attached to the cuff wherein when the cuff is positioned around the arm and the appendage is positioned on the elbow of the patient, the measurement end of the tube is forced into a position relative to a brachial artery of the patient to influence an accurate measurement of blood pressure.

2. The apparatus of claim 1, wherein the appendage comprises an indented region formed to complementarily receive the elbow of a patient.

3. The apparatus of claim 1, wherein the appendage comprises a cutout region formed to complementarily receive the elbow of a patient.

4. The apparatus of claim 1, wherein the appendage is affixed in the position relative to the cuff to fit upon the elbow of the patient based upon anthropometric data that enables accurate placement of the blood pressure cuff for a significant number of patients.

5. The apparatus of claim 1, wherein the appendage is affixed in the position relative to the cuff to fit upon the elbow of a patient based upon a one-time placement to accommodate an arm size of the patient.

6. The apparatus of claim 1, wherein when the appendage is placed over the elbow, positioning of the bottom edge of the cuff around the arm is substantially one inch above the elbow.

7. The apparatus of claim 1, wherein the appendage is affixed in the position relative to the cuff to fit upon the elbow of a patient in accordance a predetermined size calculated based upon at least one of upper arm circumference, upper arm diameter or arm length.

8. The apparatus of claim 1, wherein the appendage is one of the following:
detachably affixed to the cuff; and
permanently affixed to the cuff.

9. An apparatus for ensuring a positioning of a blood pressure cuff on an arm of a patient to affect an accurate measurement of blood pressure by a blood pressure monitor used in conjunction with the blood pressure cuff, the apparatus comprising:
an appendage affixed to the cuff and extended from an edge of the cuff between opposite ends of the cuff, the appendage affixed in a position relative to the cuff and formed in shape to complementarily receive an elbow of the patient in a manner that ensures that one or more elements of the cuff are forced in a position that enables accurate measurement of blood pressure;
wherein the elements of the cuff include a tube having a measuring end to be placed substantially near a brachial artery of the patient in order for the blood pressure monitor to accurately measure the blood pressure of the patient.

10. The apparatus of claim 9, wherein the appendage comprises at least one of the following:
an indented region formed to complementarily receive the elbow of the patient; and
a cutout region formed to complementarily receive the elbow of the patient.

11. The apparatus of claim 9, wherein the appendage further comprises at least one strap to assist in holding the appendage in place with respect to the elbow.

12. The apparatus of claim 9, wherein the appendage is affixed in the position relative to the cuff to fit upon the elbow of a patient based upon anthropometric data that enables accurate placement of the blood pressure cup for a significant number of patients.

13. The apparatus of claim 9, wherein the appendage is affixed in the position relative to the cuff to fit upon the elbow of a patient based upon a one-time placement to accommodate an arm size of the patient.

14. The apparatus of claim 9, wherein when the appendage is placed over the elbow, positioning of the bottom edge of the cuff around the arm is substantially one inch above the elbow.

15. The apparatus of claim 9, wherein the appendage is permanently affixed to the cuff.

16. The apparatus of claim 9, wherein the appendage is detachably affixed to the cuff.

17. A method for ensuring a position of a blood pressure cuff on an arm of a patient to affect an accurate measurement of blood pressure, the method comprising:
providing a blood pressure cuff apparatus comprising:
an elongated air bag of predetermined length disposed in the blood pressure cuff and adapted to be inflated the length having opposite ends to be placed around the arm of the patient, an appendage affixed to the cuff and extended from a bottom edge of the cuff between the opposite ends, the appendage affixed in a position relative to the cuff and formed in shape to fit upon an elbow of the patient in a manner that ensures that the cuff is positioned on the arm of the patient to affect an accurate measurement of the blood pressure, and a tube having a measuring end attached to the cuff wherein when the cuff is positioned around the arm and the appendage is positioned on the elbow of the patient, the measuring end of the tube is forced into position substantially near a brachial artery of the patient;
placing the appendage over the elbow of the patient wherein the appendage complimentarily receives the elbow; and
placing the cuff around the arm of the patient;
thereby ensuring proper placement of the cuff to affect an accurate measurement of blood pressure.

18. The method of claim 17, wherein placing the appendage over the elbow comprises placing the elbow in an indented region of the appendage formed to complementarily receive the elbow of the patient.

19. The method of claim 17, wherein placing the appendage over the elbow comprises placing the elbow in a cutout region of the appendage formed to complementarily receive the elbow of the patient.

20. The method of claim 17, wherein when the appendage is placed over the elbow, placing the cuff around the arm comprises positioning the bottom edge of the cuff around the arm substantially one inch above the elbow.
